(19) United States
(12) Patent Application Publication
(10) Pub. No.: US 2025/0173334 A1
(43) Pub. Date: May 29, 2025

(54) SUBJECT MONITORING

(71) Applicant: BRITISH TELECOMMUNICATIONS PUBLIC LIMITED COMPANY, London Greater London (GB)

(72) Inventors: Matthew WALLWORK, London (GB); Claudia CRISTINA, London (GB); Nektarios GEORGALAS, London (GB)

(21) Appl. No.: 18/851,014

(22) PCT Filed: Feb. 7, 2023
(86) PCT No.: PCT/EP2023/052942

§ 371 Date: Sep. 25, 2024

(30) Foreign Application Priority Data
Mar. 29, 2022 (GB) 2204451.5

(51) Int. Cl. G06F 16/2453

(52) U.S. Cl. CPC G06F 16/24539

(57) ABSTRACT

A system, method and computer program for monitoring a subject are provided. The system comprises one or more sensors configured to collect data relating to the subject. The system further comprises a query pattern store configured to store one or more query patterns. Each query pattern is associated with a respective data processor for generating summary data based on the data, the summary data conveying less information about the subject than the data. The system further comprises a query processor configured to: receive a query regarding the subject from a user of the system; identify a query pattern that matches the query; determine whether the user is permitted to make queries matching that query pattern; and in response to determining that the user is permitted, provide a response to the query, the response comprising summary data generated using the respective data processor for that query pattern.

### FIELD OF THE INVENTION

The present invention relates to the monitoring of a subject. In particular, it relates to providing data about a monitored subject in response to user queries.

### BACKGROUND TO THE INVENTION

The Internet of Things (IoT) concept extends the idea of networking general-purpose computers to the networking of smaller more single-purpose (or at least limited-purpose) devices, such as sensors and actuators. Some IoT devices are intended to be installed (or situated) within a particular environment (such as a home). Once installed they may remain static (or at least relatively so) and provide sensed data about the environment (or part thereof) in which they are installed. Other IoT devices may be wearable devices, which can be worn by their owner. Such IoT devices are intended to be mobile, accompanying the wearer and providing sensed data about the wearer or their immediate surroundings at any given point in time.

There are a wide range of different types of IoT devices, which is increasing over time. For example, there are IoT-enabled versions of doorbells, video cameras, lights, air quality monitors, temperature monitors, thermostats, noise monitors, motion sensors (e.g. PIR sensors), contact sensors (e.g. for doors and windows), pressure sensors, health monitoring devices (such as scales, blood pressure monitors, heart rate monitors, thermometers, sleep trackers, activity monitors) and so on.

There are also various IoT devices that serve to provide IoT functionality for non-IoT devices. For example, IoT-enabled sockets and IoT-enabled light switches may provide (at least some degree of) IoT functionality to electrical appliances and household lighting. Similarly, there are IoT-enabled tags (or smart tags) that may be attached to a non-IoT item (such as a wallet or keys) to allow the location of that item to be tracked.

With the proliferation of available IoT devices, it is increasingly desired to make use of IoT devices to monitor a subject (i.e. person). For example, someone living on their own, who is perhaps frail or infirm, might allow other people, such as a doctor, a carer and/or a family member, to access data from such IoT devices to allow them to check up on their wellbeing, so that they can provide suitable assistance when needed. As another example, an adolescent might permit a responsible adult (e.g. a parent or guardian) access to data from their wearable or portable IoT devices to allow their carer to monitor their activities to help protect them from danger whilst allowing them more freedom. Indeed, there are many other scenarios in which IoT devices may be beneficially employed to monitor a person.

### SUMMARY OF THE INVENTION

Whilst the use of IoT devices to allow a subject to be monitored is generally considered to be beneficial, there can be a concern that the amount of information that can be derived from the data provided by various IoT devices is more invasive of the subject's privacy than they would wish. For example, someone might permit others to access data from a sleep tracking mat so that they can check in on them in the morning to see that they have got out of bed. If they did not get out of bed (e.g. due to illness), those other people would be able to find out and provide suitable assistance. However, whilst someone might be happy with others knowing that they have got out of bed in the morning, they might be less happy about sharing other information that could also be deduced from the data provided by the sleep tracking mat. For example, the data might allow the other people to determine how long they had slept for, what time they went to bed, what time they got up, the number of times they got out of bed during the night, how restful their sleep was and so on. This extra information that can be deduced from the data provided by the IoT device(s) (e.g. the sleeping mat) is superfluous to the information that the subject actually wishes to share, namely whether they got out of bed that morning.

It would be desirable to provide a mechanism for allowing a subject to be monitored via network-connected sensors (such as provided by IoT devices) in such a way that they can better control the information that is shared. That is to say, a mechanism which allows a subject to maintain their privacy by preventing the unintended sharing of information that is superfluous to that which they wish to share.

In a first aspect of the present invention, there is provided a system for monitoring a subject, the system comprising: one or more sensors configured to collect data relating to the subject; a query pattern store configured to store one or more query patterns, each query pattern being associated with a respective data processor for generating summary data based on the data, the summary data conveying less information about the subject than the data; and a query processor configured to: receive a query regarding the subject from a user of the system; identify a query pattern that matches the query; determine whether the user is permitted to make queries matching that query pattern; and in response to determining that the user is permitted, provide a response to the query, the response comprising summary data generated using the respective data processor for that query pattern.

At least one of the query patterns may indicate one or more respective parameters to be extracted from queries that match that query pattern. The respective data processor associated with that query pattern may be configured to generate the summary data based on the one or more respective parameters. The query processor may be further configured to extract the one or more respective parameters from the query in response to the query matching the at least one of the query patterns and provide the parameters to the respective data processor for that query pattern when generating the summary data.

The system may further comprise a query suggestion module configured to suggest one or more queries to the user of the system that match query patterns that the user is permitted to make.

The query processor may be further configured to carry out one or more predetermined actions in response to determining that the user is not permitted to make queries matching the query pattern. The one or more predetermined actions comprise one or more, or all, of: notifying the subject of the query; logging the request; and suggesting an alternative query to the user based on the query and the query patterns that the user is permitted to make.

The determination as to whether the user is permitted to make queries matching a query pattern may be based, at least in part, on a role assigned to the user.

The subject may be an occupant in a domestic environment that the one or more sensors are configured to monitor.

In a second aspect of the present invention, there is provided a computer implemented method for providing data about a subject being monitored, the method comprising: receiving a query regarding the subject from a user; identifying a query pattern that matches the query, wherein each query pattern is associated with a respective data processor for generating summary data based on data relating to the subject collected by one or more sensors, the summary data conveying less information about the subject than the data collected by the sensors; determining whether the user is permitted to make queries matching that query pattern; and in response to determining that the user is permitted, providing a response to the query, the response comprising summary data generated using the respective data processor for that query pattern.

The query pattern may indicate one or more respective parameters to be extracted from queries that match that query pattern and the respective data processor associated with the query pattern may be configured to generate the summary data based on the one or more respective parameters. The method may further comprise extracting the one or more respective parameters and providing the parameters to the respective data processor for the query pattern for use in generating the summary data.

The method further comprises suggesting one or more queries to the user of the system that match query patterns that the user is permitted to make.

The method may further comprise carrying out one or more predetermined actions in response to determining that the user is not permitted to make queries matching the query pattern. The one or more predetermined actions comprise one or more, or all, of: notifying the subject of the query; logging the request; and suggesting an alternative query to the user based on the query and the query patterns that the user is permitted to make.

The determination as to whether the user is permitted to make queries matching a query pattern may be based, at least in part, on a role assigned to the user.

The subject may be an occupant in a domestic environment that the one or more sensors are configured to monitor.

The use of query patterns enables control over access to the subject's information. Each query pattern can be associated with a particular intent behind the monitoring (e.g. to determine that a specific event has occurred, such as whether the “got out of bed” event has occurred for the subject). The respective data processor that is associated with each query pattern enables a response to the query be generated from the available sensor data that provides sufficient information to satisfy the intent of the query without providing any superfluous information that would also have been conveyed by the raw data.

In a third aspect of the present invention, there is provided a computer program which, when executed by one or more processors, is arranged to carry out a method according to the second aspect.

### BRIEF DESCRIPTION OF THE FIGURES

Embodiments of the present invention will now be described by way of example only, with reference to the accompanying drawings, in which:

FIG. 1 is a block diagram of a computer system suitable for the operation of embodiments of the present invention.

FIG. 2 schematically illustrates a system for monitoring a subject according to embodiments of the invention.

FIG. 3 is a flowchart representing a method for providing data about a subject being monitored.

### DETAILED DESCRIPTION OF EMBODIMENTS

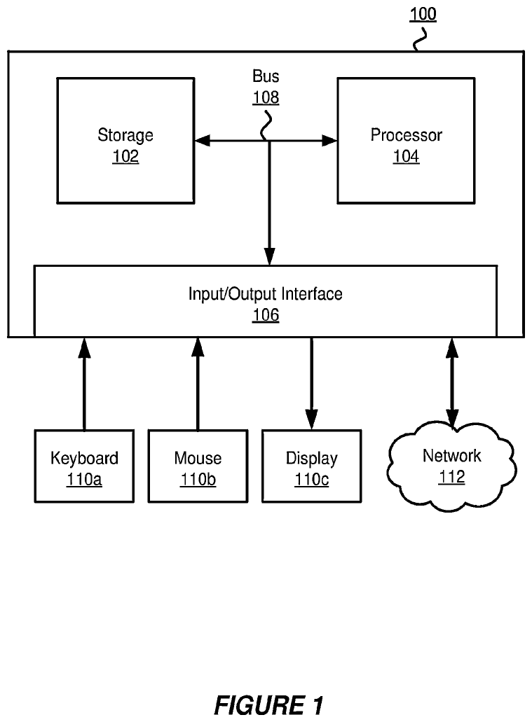

FIG. 1 is a block diagram of a computer system 100 suitable for the operation of embodiments of the present invention. The system 100 comprises: a storage 102, a processor 104 and an input/output (I/O) interface 106, which are all communicatively linked over one or more communication buses 108.

The storage (or storage medium or memory) 102 can be any volatile read/write storage device such as a random access memory (RAM) or a non-volatile storage device such as a hard disk drive, magnetic disc, optical disc, ROM and so on. The storage 102 can be formed as a hierarchy of a plurality of different storage devices, including both volatile and non-volatile storage devices, with the different storage devices in the hierarchy providing differing capacities and response times, as is well known in the art.

The processor 104 may be any processing unit, such as a central processing unit (CPU), which is suitable for executing one or more computer programs (or software or instructions or code). These computer programs may be stored in the storage 102. During operation of the system, the computer programs may be provided from the storage 102 to the processor 104 via the one or more buses 108 for execution. One or more of the stored computer programs, when executed by the processor 104, cause the processor 104 to carry out a method according to an embodiment of the invention, as discussed below (and accordingly configure the system 100 to be a system 100 according to an embodiment of the invention).

The input/output (I/O) interface 106 provides interfaces to devices 110 for the input or output of data, or for both the input and output of data. The devices 110 may include user input interfaces, such as a keyboard 110a or mouse 110b as well as user output interfaces such as a display 110c. Other devices, such a touch screen monitor (not shown) may provide means for both inputting and outputting data. The input/output (I/O) interface 106 may additionally or alternatively enable the computer system 100 to communicate with other computer systems via one or more networks 112. It will be appreciated that there are many different types of I/O interface that may be used with computer system 100 and that, in some cases, computer system 100 may include more than one I/O interface. Furthermore, there are many different types of device 110 that may be used with computer system 100. The devices 110 that interface with the computer system 100 may vary considerably depending on the nature of the computer system 100 and may include devices not explicitly mentioned above, as would be apparent to the skilled person. For example, in some cases, computer system 100 may be a server without any connected user input/output devices. Such a server may receive data via a network 112, carry out processing according to the received data and provide the results of the processing via a network 112.

It will be appreciated that the architecture of the system 100 illustrated in FIG. 1 and described above is merely exemplary and that other computer systems 100 with different architectures (such as those having fewer components, additional components and/or alternative components to those shown in FIG. 1) may be used in embodiments of the invention. As examples, the computer system 100 could comprise one or more of: a personal computer; a laptop; a tablet; a mobile telephone (or smartphone); a television set (or set top box); a games console; an augmented/virtual reality headset; a server; or indeed any other computing device with sufficient computing resources to carry out a method according to embodiments of this invention.

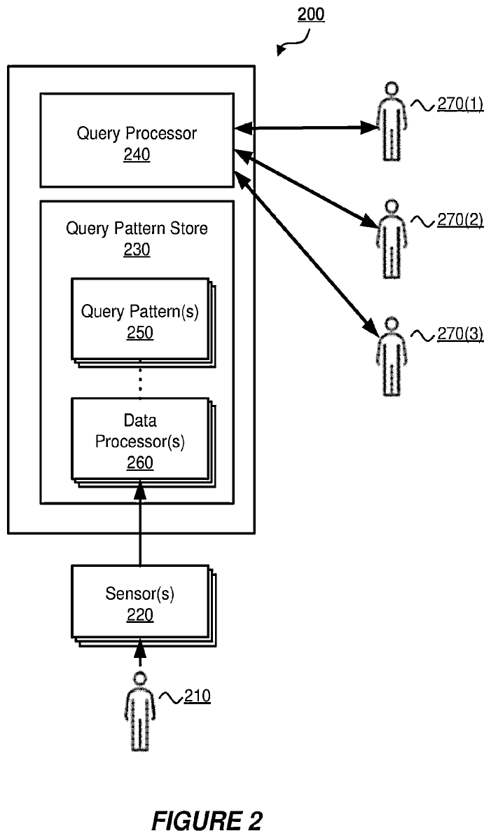

FIG. 2 schematically illustrates a system 200 for monitoring a subject 210 according to embodiments of the invention. The system 200 comprises one or more sensors 220, a query pattern store 230 and a query processor 240.

The one or more sensors 220 are configured to collect data relating to the subject. This data may be collected from the sensor data provided by one or more IoT devices, such as any of the example IoT devices described above. In some cases, the one or more sensors may include sensors embedded within one or more IoT devices may be installed within an environment in which the subject is present at least some of the time. For example, IoT devices may be installed in a domestic environment where the subject 210 lives. Additionally or alternatively, the one or more sensors 220 may include sensors embedded within one or more wearable IoT devices that are associated with the subject 210. The data collected about the subject 210 by the sensors is made available for use by the other components of system 200.

The query pattern store 230 is configured to store one or more query patterns 250. For example, a storage container, such as a database, may be defined within a storage 102 of a computer system 100 that is providing system 200 (or part thereof).

The query patterns 250 define different monitoring intents. That is to say, each query pattern is associated with a particular item of information about the subject 210 that may be monitored. These intents can best be thought of as the types of questions that may be asked about the subject. For example, the query patterns 250 may reflect questions such as “Has the subject 210 woken up this morning?”, “How long did the subject 210 sleep for?” or “What time did the subject 210 get up this morning?”.

Each of the query patterns 250 is associated with a respective data processor 260. The respective data processor 260 associated with a particular query pattern 250 comprises logic for generating data that addresses the monitoring intent associated with that query pattern 250 (i.e. for answering the question posed by that intent). The data processor(s) 260 generate the data to address the monitoring intent from the data that is collected by the sensors 220. The generated data conveys less information about the subject than the full data collected by the sensors 220 and so may be referred to herein as summary data.

Ideally, the logic for each data processor 260 only conveys sufficient information to fully address the monitoring intent for its associated query pattern 250 (i.e. to answer the question posed by that intent) and no more. For example, where the query pattern 250 reflects a monitoring intent of “Has the subject 210 woken up this morning?”, the summary data generated by the associated data processor 260 may be a simple Boolean “Yes” or “No” value. Similarly, where the query pattern 250 reflects a monitoring intent of “How long did the subject 210 sleep for?”, the summary data generated by the associated data processor 260 may be a numerical value representing at time period (e.g. an integer value representing a number of minutes spent sleeping). As a further example, where the query pattern 250 reflects a monitoring intent of “What time did the subject 210 get up this morning?”, the summary data generated by the associated data processor 260 may be a representation of a time (e.g. an integer value representing the number of minutes after midnight that the subject 210 woke up). Accordingly, by processing the data to generate more specific summary data that addresses a particular monitoring intent, the data processors 260 discard (or filter) out other information that could be derived from the raw data collected from the sensors 220.

Any suitable technique for implementing logic that enables the summary data to be generated from the data collected from the sensors 220 may be used. For example, a software function may be defined which contains the necessary logic to generate the summary data for addressing a particular monitoring intent defined by a query pattern 250. These functions can then be invoked (or executed) in order to generate the summary data for responding to queries following that query pattern 250. Such functions may be stored in the query pattern store 230 in association with their respective query pattern 250 to allow for their retrieval and execution as and when needed. Additionally, or alternatively, these functions may be provided as a service remotely from the system 200. In such cases, the functions may be invoked remotely, such as by issuing a remote procedure call (RPC), as will be familiar to the skilled person.

The query processor 240 is configured to respond to queries about the subject 210 being monitored from one or more users 270 of the system 200.

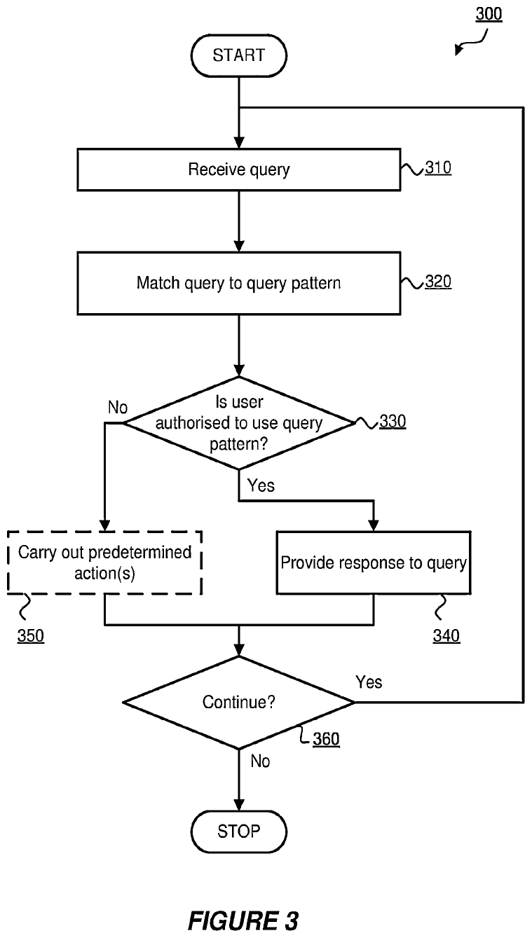

The operation of the system 200 the query processor 240 will be discussed further in conjunction with FIG. 3, which is a flowchart representing a method 300 for providing data about the subject 210 being monitored. The query processor 240 is configured to operate according to this method 300 to respond to queries about the subject 210.

At an operation 310, the method 300 receives a query regarding the subject 210 from one of the users 270 of the system. The method 310 then proceeds to an operation 320.

At operation 320, the method 300 identifies a query pattern 250 that matches the received query. It will be appreciated that there are many different ways that a query can be matched to a query pattern 250 and that any suitable technique may be used. In the simplest cases, the received query may indicate the specific query pattern 250 to which it relates, in which case operation 320 simply uses that indication to identify the query pattern. In other cases, the system 200 may provide a more conversational style of interface in which the users 270 may express the query natural language in the form of a question about the subject 210. In such cases, various natural language processing techniques may be used to identify which of the query patterns 250 in the query pattern store 230 the query relates to. For example, machine learning may be used to generate a model which classifies a query into the query patterns 250 to which it relates. Alternatively, a more rules based approach may be used, such as by using regular expressions to parse the query. For example, each query pattern 250 may be associated with one or more regular expressions and the received query may be evaluated against those regular expressions to determine whether it matches that query pattern. Although not shown in FIG. 3, if the query does not match any of the query patterns 250 in the query pattern store 230, the method 200 may provide an error message to the user 270 that issued the query and processing may skip to operation 360. Otherwise, having identified a query pattern 250 that matches the received query, the method 300 proceeds to an operation 330.

At operation 330, the method 300 determines whether the user 270 is permitted (or authorised) to make queries matching the query pattern 250 that was identified at operation 320.

In some cases, the authorisation may be implicit. That is to say, through the inclusion of a particular query pattern 250 in the query pattern store 230, it is implied that any user 270 of the system 200 can make queries according to that pattern (assuming that that they are properly authenticated and authorised to use the system 200).

In other cases, each of the query patterns 250 may be associated with an indicator as to whether queries according to that pattern are permitted to be made by the users 270 of the system 200. This can allow individual query patterns 250 to be allowed or disallowed as desired. These indicators may be generic or user specific. That is to say, they may apply to all users or to a specific user. For example, an indicator may specify that a first query pattern 250 may be used by any user, whilst a second query pattern 250 may only be used by particular user(s), such as a first user 270(1). Accordingly, whilst any of the users 270(1), 270(2) and 270(3) may be authorised to make queries according to the first query pattern 250, only the first user 270(1) may be authorised to make queries according to the second query pattern 250. Any queries according to the second query pattern 240 made by the second or third users 270(2) and 270(3) may therefore be considered to be unauthorised.

In yet other cases, the users 270 may each by associated with one or more roles (or profiles) and a role-based authentication scheme may be used relative to the query patterns 250. For example, the first user 270(1) may be assigned the role of ‘Carer’, the second user 270(2) may be assigned the role of ‘Medical Professional’ and the third user may be assigned the role of ‘Family’. The query patterns 250 may then each be associated with a respective indication of the roles that are permitted to make queries according to that query pattern 250. Accordingly, the determination as to whether the user is permitted to make queries matching a particular query pattern 250 is based on the role assigned to the user. That is to say, whether the user has been assigned a role that is permitted to make queries according to that query pattern 250.

In yet other cases, each of the query patterns 250 may be associated with a privacy impact score indicating the level of sensitivity that the information provided by responses to such queries is considered to have. A permitted sensitivity level may be assigned to each user and used to control whether or not they are permitted to make queries according to particular query patterns. Similarly, where the users 270 are associated with roles, a permitted sensitivity level may additionally or alternatively be assigned to each role. Where there are multiple permitted sensitivity levels associated with a user 270, such as where a permitted sensitivity level is associated with the user 270 individually as well as with one or more roles that the user is associated with, the highest permitted sensitivity level may be used for that user 270. Alternatively, any individually specified permitted sensitivity level may be considered to override any more general sensitivity levels that are associated with a user's roles. The determination as to whether a user 270 is permitted to make queries according to a particular query pattern 250 may therefore be based on the privacy impact score for the query pattern 250 and the permitted sensitivity level assigned to the user 270 (i.e. the user 270 is only permitted to make queries according to that pattern 250 if their permitted sensitivity level exceeds the privacy impact score of the query pattern 250).

In yet other cases, the determination as to whether the user 270 is permitted (or authorised) to make queries matching the query pattern may be based, at least in part, on a classification of the type of information that is provided by the response to such queries. That is to say, each query pattern 250 may be associated with a particular type of information (e.g. “sleep”, “weight”, “nutrition”, etc.) and each user 270 may be associated with one or more types of information that they are permitted to make queries about. Accordingly, a user 270 may only be permitted to make a query according to a query pattern 250 if they are permitted to make queries about the type of information that is provided by response to queries according to that query pattern 250.

Of course, it will be appreciated that these different approaches can be combined in various different ways to achieve more complex and fine-grained authorisation schemes to achieve the desired level of control over the permission to make different types of query.

In some cases, the system 200 may comprise an authentication learning module (not shown), which is configured to learn an appropriate set of permissions for each user. This module learns the appropriate set of permissions based on queries that are made by a user 270 during a training period. For example, when a user is setup on the system 200, they may access the system under a training mode in which all of their queries are logged. At the end of the training mode, the queries that were made may be reviewed (e.g. by the monitored subject 210) and an indication as to whether that query should be allowed or refused may be recorded against each query. This data may then be used by the authentication learning module to deploy an appropriate set of permissions for that user. For example, the authentication learning module may identify a role, permitted sensitivity level and/or permitted data types for the user that best fits the training data.

If it is determined at operation 330 that the user 270 is permitted to make queries matching the query pattern 250 that was identified at operation 320, the method 300 proceeds to an operation 340. Otherwise, if the user 270 is not permitted to make queries according to that query pattern 250, the method 300 proceeds to an optional operation 350.

At operation 340, the method 300 provides a response to the query. Specifically, the method 300 uses the respective data processor 260 for the query pattern 250 that was identified as matching the received query to generate the summary data and uses the summary data as a response to the query.

In some cases, the query pattern 250 may indicate one or more parameters that are to be extracted from the received query for use by the data processor 260 in generating the summary data. For example, where the query pattern 250 reflects a monitoring intent of “Did the subject 210 have breakfast?” the query pattern may indicate that a parameter indicating a particular day that the query relates to should be present in the query, such as “Did the subject 210 have breakfast today?” or “did the subject 210 have breakfast yesterday?”. Accordingly, the method 300 may extract the appropriate parameters from the query and provide them to the data processor 260 to allow the data processor 260 to prepare the appropriate summary data (e.g. the summary data for the particular day in question). This can be achieved by using natural language processing techniques to extract the parameters from the query in much the same way as discussed in relation to operation 320 for matching queries to query patterns.

In some cases, the system 200 and method 300 may operate according the publish-subscribe messaging pattern (otherwise referred to as pub/sub). In which case, the method 300 may register the user 270 as a subscriber for that query pattern 250 and subsequently send updates to the user 270 as appropriate.

Having provided a response to the query at operation 340, the method proceeds to an operation 360.

At optional operation 350, the method 300 carries out (or causes to be carried out) one or more predetermined actions. For example, the method 300 may log the query and/or notify the subject 210 (or an administrative entity) of the query. This can allow the types of information that is being requested to be monitored and for the permissions to be adjusted if desired.

In some cases, the method 300 may determine a difference between a permission level needed for the query pattern 250 associated with the query and a permission level associated with the requesting user. For example, where each query pattern 250 is associated with a privacy impact score and the user is assigned with a permitted sensitivity level, the difference may be the difference between the user's permitted sensitivity level and the privacy impact score associated with the query pattern 250. In such cases, the performance of the predetermined action may be dependent upon this difference. For example, the predetermined action(s) may only be taken if the difference is above some predetermined level. As another example, the predetermined action(s) may be selected based on the difference such that a first set of actions is taken if the difference is below a predetermined threshold and a second set of actions is taken otherwise.

In some cases, the method may monitor the query patterns 250 that are used by each user 270 to establish a normal pattern of querying for each user. For example, where the query patterns 250 are associated with a type of information that is provided in response to queries according to those patterns, the method 300 may establish a normal pattern of data types that are queried by each user. Accordingly, the performance of the predetermined action(s) may be dependent upon how different the received query is from those that are typically made by that user 270. For example, the predetermined action(s) may only be taken if the query is determined to be unusual for the user 270. As another example, the predetermined action(s) may be selected based on how normal the query is for the user 270. For example, a first set of actions may be taken if the query is determined to be normal for the user, whilst a second set of actions may be taken otherwise.

At operation 360, the method 300 determines whether to continue processing queries about the subject 210. In general, it is expected that the method 300 will be run continuously in an ongoing manner so that it can continue to respond to queries at any point in time (although this need not be the case). Accordingly, at operation 360, if it is determined that processing should continue, the method 300 reiterates back to operation 310 ready to receive a further query. Otherwise, the method 300 ends.

Although not shown in FIG. 2, the system 200 may further comprise a query suggestion module. This module may be configured to suggest queries 270 to the users of the system 200. These suggestions may be produced proactively (e.g. prior to receipt of a query from the user). The query suggestion module may identify the query patterns 250 that a user 270 would be permitted to make and make suggest queries to the user that accord to those query patterns 250. The suggestions may also (or alternatively) be produced reactively (e.g. following receipt of a query from the user). That is to say, if it is determined at operation 330 of the method 300 performed by the query processor 240 that the user 270 is not permitted to make a certain query, suggestions may be made as to similar queries that the user 270 would be permitted to make. For example, the original query made by a user may be identified as matching a query pattern 250 that is associated with a particular type of information (e.g. “sleep” information). Although the user 270 may not be permitted to make their original query, the query suggestion module can search for other query patterns 250 that are associated with the same type of data that the user 270 would be permitted to make and can suggest queries to the user that follow those query patterns 250.

Throughout this description, the system 200 and method 300 have been described as monitoring a singular subject 210. However, it will be appreciated that the system 200 and method 300 can readily be adapted for monitoring multiple subjects 210, such as multiple occupants in a domestic environment. In such cases, the queries may indicate an identity of the subject to which the query relates. This identity may be used in determining authorisations to make different queries (i.e. different permissions may relate to the query patterns 250 in respect of the different subjects 210) and may be used by the data processors 260 to generate a response for the correct subject 210.

Similarly, although the system 200 has been illustrated as having three users 270(1), 270(2) and 270(3), it will be appreciated that this is just an example and that other numbers of users may be used instead.

Insofar as embodiments of the invention described are implementable, at least in part, using a software-controlled programmable processing device, such as a microprocessor, digital signal processor or other processing device, data processing apparatus or system, it will be appreciated that a computer program for configuring a programmable device, apparatus or system to implement the foregoing described methods is envisaged as an aspect of the present invention. The computer program may be embodied as source code or undergo compilation for implementation on a processing device, apparatus or system or may be embodied as object code, for example. Suitably, the computer program is stored on a carrier medium in machine or device readable form, for example in solid-state memory, magnetic memory such as disk or tape, optically or magneto-optically readable memory such as compact disk or digital versatile disk etc., and the processing device utilises the program or a part thereof to configure it for operation. The computer program may be supplied from a remote source embodied in a communications medium such as an electronic signal, radio frequency carrier wave or optical carrier wave. Such carrier media are also envisaged as aspects of the present invention. It will be understood by those skilled in the art that, although the present invention has been described in relation to the above described example embodiments, the invention is not limited thereto and that there are many possible variations and modifications which fall within the scope of the invention. The scope of the present invention includes any novel features or combination of features disclosed herein. The applicant hereby gives notice that new claims may be formulated to such features or combination of features during prosecution of this application or of any such further applications derived therefrom. In particular, with reference to the appended claims, features from dependent claims may be combined with those of the independent claims and features from respective independent claims may be combined in any appropriate manner and not merely in the specific combinations enumerated in the claims.

1. A system for monitoring a subject, the system comprising:
one or more sensors configured to collect data relating to the subject;
a query pattern store configured to store one or more query patterns, each query pattern being associated with a respective data processor for generating summary data based on the data, the summary data conveying less information about the subject than the data; and
a query processor configured to:
receive a query regarding the subject from a user of the system;
identify a query pattern that matches the query;
determine whether the user is permitted to make queries matching that query pattern; and
in response to determining that the user is permitted, provide a response to the query, the response comprising summary data generated using the respective data processor for that query pattern.

2. The system of claim 1, wherein:
at least one of the query patterns indicates one or more respective parameters to be extracted from queries that match that query pattern;
the respective data processor associated with that query pattern is configured to generate the summary data based on the one or more respective parameters; and
the query processor is further configured to extract the one or more respective parameters from the query in response to the query matching the at least one of the query patterns and provide the parameters to the respective data processor for that query pattern when generating the summary data.

3. The system of claim 1 further comprising a query suggestion module configured to suggest one or more queries to the user of the system that match query patterns that the user is permitted to make.

4. The system of claim 1, wherein the query processor is further configured to carry out one or more predetermined actions in response to determining that the user is not permitted to make queries matching the query pattern.

5. The system of claim 3, wherein the one or more predetermined actions comprise one or more, or all, of:
notifying the subject of the query;
logging the request; and
suggesting an alternative query to the user based on the query and the query patterns that the user is permitted to make.

6. The system of claim 1, wherein the determination as to whether the user is permitted to make queries matching a query pattern is based, at least in part, on a role assigned to the user.

7. The system of claim 1, wherein the subject is an occupant in a domestic environment that the one or more sensors are configured to monitor.

8. A computer implemented method for providing data about a subject being monitored, the method comprising:
receiving a query regarding the subject from a user;
identifying a query pattern that matches the query, wherein each query pattern is associated with a respective data processor for generating summary data based on data relating to the subject collected by one or more sensors, the summary data conveying less information about the subject than the data collected by the sensors;
determining whether the user is permitted to make queries matching that query pattern; and
in response to determining that the user is permitted, providing a response to the query, the response comprising summary data generated using the respective data processor for that query pattern.

9. The method of claim 8, wherein the query pattern indicates one or more respective parameters to be extracted from queries that match that query pattern and the respective data processor associated with the query pattern is configured to generate the summary data based on the one or more respective parameters, the method further comprising:
extracting the one or more respective parameters and providing the parameters to the respective data processor for the query pattern for use in generating the summary data.

10. The method of claim 8, wherein the method further comprises suggesting one or more queries to the user of the system that match query patterns that the user is permitted to make.

11. The method of claim 8, further comprising carrying out one or more predetermined actions in response to determining that the user is not permitted to make queries matching the query pattern.

12. The method of claim 11, wherein the one or more predetermined actions comprise one or more, or all, of:
notifying the subject of the query;
logging the request; and
suggesting an alternative query to the user based on the query and the query patterns that the user is permitted to make.

13. The method of claim 8, wherein the determination as to whether the user is permitted to make queries matching a query pattern is based, at least in part, on a role assigned to the user.

14. The method of claim 8 wherein the subject is an occupant in a domestic environment that the one or more sensors are configured to monitor.

15. A computer program which, when executed by one or more processors, is arranged to carry out a method according to claim 8.
